SEQUENCE LISTING (7 sequences)

SEQ ID NO: 1 - protein, 25 aa, Crassostrea gigas
  VARIANT (3): an S or R residue
TAXNEANVNIYLHLSDDEDSNYENS

SEQ ID NO: 2 - protein, 14 aa, Crassostrea gigas
EPNAFMPGNLHHRV

SEQ ID NO: 3 - protein, 10 aa, Crassostrea gigas
  VARIANT (4): a C residue
EHGXDTIGEL

SEQ ID NO: 4 - protein, 174 aa, Perna canaliculus
  VARIANT (3): an S or R residue
TAXNEANVNIYLHLSDDEDSNYENSMHYAQCEMEPNAFMPGNLHHRVHGSIEMHQRGDGPLEMSFCLSGFNVSEDFADHNHGLQIHEYGDMEHGCDTIGELYHNEHAPNHDNPGDLGDLHDDDHGNVDATRTFDWLTIGHTDGILGRSLAILQGDHTSHTAVIACCVIGRSHAH

SEQ ID NO: 5 - DNA, 606 bp, Crassostrea gigas
  polyA_signal (601)..(606)
  misc_feature (526)..(528)
atgactgctagtaatgaagctaatgttaacatttatcttcacctttctgatgatgaagattccaactacgaaaactccatgcattatgctcaatgcgagatggaacccaatgcctttatgccgggcaacctccaccatagggtccatggaagcatcgaaatgcatcaacggggagacggacctttggaaatgagcttctgtctgtccggattcaacgtcagtgaagactttgctgatcacaaccacggacttcagatccacgagtacggagatatggaacatggctgtgacaccattggagaactgtaccataatgagcacgcccccaaccacgataaccccggtgacctcggagatctccatgacgacgaccacggaaatgtggatgctaccaggactttcgattggctcaccatcggacatacagacggaattcttggccgatcattggctattctccagggagaccacacctctcataccgctgtcatcgcttgctgcgtcattggtcgctctcatgcccactagatgatcataacggaccattctaaataaaagattatcatttatatcgaacttcagtagaaataaaaacttacaaaaaaa

SEQ ID NO: 6 - DNA, 528 bp, Crassostrea gigas
  CDS (1)..(528)
atgactgctagtaatgaagctaatgttaacatttatcttcacctttctgatgatgaagattccaactacgaaaactccatgcattatgctcaatgcgagatggaacccaatgcctttatgccgggcaacctccaccatagggtccatggaagcatcgaaatgcatcaacggggagacggacctttggaaatgagcttctgtctgtccggattcaacgtcagtgaagactttgctgatcacaaccacggacttcagatccacgagtacggagatatggaacatggctgtgacaccattggagaactgtaccataatgagcacgcccccaaccacgataaccccggtgacctcggagatctccatgacgacgaccacggaaatgtggatgctaccaggactttcgattggctcaccatcggacatacagacggaattcttggccgatcattggctattctccagggagaccacacctctcataccgctgtcatcgcttgctgcgtcattggtcgctctcatgcccactag

SEQ ID NO: 7 - protein, 175 aa, Crassostrea gigas
MTASNEANVNIYLHLSDDEDSNYENSMHYAQCEMEPNAFMPGNLHHRVHGSIEMHQRGDGPLEMSFCLSGFNVSEDFADHNHGLQIHEYGDMEHGCDTIGELYHNEHAPNHDNPGDLGDLHDDDHGNVDATRTFDWLTIGHTDGILGRSLAILQGDHTSHTAVIACCVIGRSHAH

(19) United States
(12) Patent Application Publication
(10) Pub. No.: US 2004/0146525 A1
(43) Pub. Date: Jul. 29, 2004

(54) Shellfish protein

(76) Inventors: Paul Douglas Scotti, Auckland (NZ); Sally Caroline Dearing, Auckland (NZ); David Roger Greenwood, Auckland (NZ)

Correspondence Address: JACOBSON HOLMAN PLLC, 400 SEVENTH STREET N.W., SUITE 600, WASHINGTON, DC, 20004, US

(21) Appl. No.: 10/472,184

(22) PCT Filed: Mar. 27, 2002
(86) PCT No.: PCT/NZ02/00044

(30) Foreign Application Priority Data
Mar. 27, 2001 (NZ) 510782
Jun. 19, 2001 (NZ) 512463

(51) Int. Cl. A61K 39/02; C07K 14/195

(52) U.S. Cl. 424/190.1; 530/400

(57) ABSTRACT

This invention provides a protein which exhibits metal ion binding activity. The protein can be readily extracted from the pacific oyster, Crassostrea gigas and can be formulated into bioremediation compositions, foodstuffs, nutraceuticals, and the like. It has a molecular weight of about 20 kDa and and amino acid sequence which includes one or more of the following:

(a) TAXNEANVNIYLHLSDDEDSNYENS (N-terminus) (SEQ ID NO:1)

(b) EPNAFMPGNLHHRV (SEQ ID NO:2)

(c) EHGXDTIGEL (SEQ ID NO:3)

This invention relates to a protein and compositions which contain it. More particularly, it relates to a protein with metal cation binding characteristics.

### BACKGROUND

Aquatic organisms such as shellfish have to cope with a wide range of pollutants in the marine environment. One of the major pollution problems is metal contamination particularly in areas where industrial activity occurs. The blue mussel (Mytilus edulis), for example, has been shown to take up cadmium from its environment and this cadmium is then transported to the kidneys by the circulatory system where it accumulates (Nair and Robinson, 2001). In the blue mussel there are a number of protein subunits that are capable of binding cadmium (Robinson et al, 1997). One of these is a histidine-rich protein with each subunit capable of binding metal ions. Such proteins, isolated and purified from shellfish, could be valuable for a number of bioremediation applications.

It is, however, difficult to obtain these proteins from mussels. Extracting from whole shellfish involves a substantial amount of purification starting with a crude homogenate in order to obtain pure or even a relatively pure preparation of protein. Alternatively, relatively pure preparations can be obtained directly from mussel haemolymph but this is a labour-intensive process requiring, for example, extraction of fluid from an adductor muscle (WO 00/39165). Neither of these processes is suitable for large-scale preparation of purified proteins.

The applicants have now identified and characterised a novel protein, from the Pacific oyster (Crassostrea gigas) which exhibits metal cation binding characteristics. This protein can be obtained easily from the oyster, in reasonable quantities, and in a relatively pure state with little processing. It is towards this protein (cavortin) that the present invention is broadly directed.

### SUMMARY OF THE INVENTION

Accordingly, in a first aspect the present invention provides a protein obtainable from Crassostrea gigas which has an apparent molecular weight of 31 kDa determined by SDS-PAGE and which has metal binding characteristics, or an active fragment thereof.

Conveniently, the protein is obtainable from the haemolymph of C. gigas.

Preferably, the protein is a self-aggregating protein.

In a further aspect, the present invention provides an isolated protein which has a molecular weight of about 31 kDa determined by SDS-PAGE and an amino acid sequence which includes one or more of the following sequences:

| | | |
|---|---|---|
| (a) | TAXNEANVNIYLHLSDDEDSNYENS | (SEQ ID NO:1) |
| | (N-terminus) | |
| (b) | EPNAFMPGNLHHRV | (SEQ ID NO:2) |
| (c) | EHGXDTIGEL | (SEQ ID NO:3) |

or an active fragment thereof.

In a further aspect, the invention provides an isolated protein which includes the amino acid sequence of:

| | |
|---|---|
| TAXNEANVNIYLHLSDDEDSNYENSMHYAQCEMEPNAFMPGNLHHRVHGSIEMHQRG | (SEQ ID NO:4) |
| DGPLEMSFCLSGFNVSEDFADHNHGLQIHEYGDMEHGCDTIGELYHNEHAPNHDNPG | |
| DLGDLHDDDHGNVDATRTFDWLTIGHTDGILGRSLAILQGDHTSHTAVIACCVIGRS | |
| HAH | |

or an active fragment thereof.

Desirably, said protein or fragment has activity as a metal ion binding agent, especially of divalent cations.

In one embodiment, said protein or fragment is metal enriched.

The invention further provides a protein which is a functionally equivalent variant of a protein or fragment as defined above.

Still further, the invention provides a protein which is obtainable from a shellfish other than C. gigas and which is a functionally equivalent variant of a protein or fragment as defined above.

The invention also provides a metal enriched protein which is a copper/zinc superoxide dismutase derived self-aggregating protein obtainable from shellfish, or a functionally equivalent variant or fragment thereof.

Also provided is a shellfish extract containing a metal enriched protein or fragment of the invention.

In another aspect, the invention provides a polynucleotide encoding a protein or fragment as defined above.

The polynucleotide may include the nucleotide sequence of:

| | |
|---|---|
| ATGACTGCTAGTAATGAAGCTAATGTTAACATTTATCTTCACCTTTCTGATGATGAAGAT | (SEQ ID NO:5) |
| TCCAACTACGAAAACTCCATGCATTATGCTCAATGCGAGATGGAACCCAATGCCTTTATG | |
| CCGGGCAACCTCCACCATAGGGTCCATGGAAGCATCGAAATGCATCAACGGGGAGACGGA | |
| CCTTTGGAAATGAGCTTCTGTCTGTCCGGATTCAACGTCAGTGAAGACTTTGCTGATCAC | |
| AACCACGGACTTCAGATCCACGAGTACGGAGATATGGAACATGGCTGTGACACCATTGGA | |
| GAACTGTACCATAATGAGCACGCCCCCAACCACGATAACCCCGGTGACCTCGGAGATCTC | |
| CATGACGACGACCACGGAAATGTGGATGCTACCAGGACTTTCGATTGGCTCACCATCGGA | |
| CATACAGACGGAATTCTTGGCCGATCATTGGCTATTCTCCAGGGAGACCACACCTCTCAT | |
| ACCGCTGTCATCGCTTGCTGCGTCATTGGTCGCTCTCATGCCCACTAGATGATCATAACG | |
| GACCATTCTAAATAAAAGATTATCATTTATATCGAACTTCAGTAGAAATAAAAACTTACA | |
| AAAAAA... (3′ poly-A terminus) | |

or a variant thereof.

Still further, the invention provides a vector or construct which includes a polynucleotide as defined above.

In another aspect, the invention provides a composition which comprises a protein or fragment as defined above.

The composition may be a medicament, a food, a dietary supplement, (optionally including the protein associated with or bound to at least one divalent cation of dietary significance) or a bioremediation agent.

In still another aspect, the invention provides a process for obtaining a protein as defined above which comprises the step of centrifuging material containing C. gigas haemolymph or an extract thereof and recovering the sedimented protein.

### DESCRIPTION OF THE DRAWINGS

While the present invention is broadly as defined above, it also includes embodiments of which the following description provides examples. In particular, a better understanding of the present invention will be gained through reference to the accompanying drawings in which

FIG. 1 is a 10% SDS-PAGE gel of self-aggregating proteins: lane 1—protein molecular weight standards (molecular mass in kDA; lane 2—oyster protein, cavortin.

FIG. 2 depicts the amino acid sequence of cavortin as inferred from the nucleotide base sequence obtained from cDNA and from direct microsequencing of CNBr cleavage fragments. Shading represents amino acid sequences obtained by microsequencing the mature cavortin molecule (N-terminus) or by microsequencing of fragments following CNBr-cleavage. Underline indicates the presumed polyadenylation signal. The N-terminal microsequence differs from the inferred sequence by an inferred “S” residue instead of the “R” amino acid residue obtained by microsequencing; this is indicated by the first bold “X” in italics. The codons for these two amino acids are AGY (for S) and AGR (for R). The blocked sequence NVS is a potential glycosylation site. The second bold “X” in italics represents an inferred “C” residue from microsequencing.

FIG. 3 shows the HPLC elution profile of C. gigas cell-free haemolymph; measurements were at 218 nm; the single peak represents the oyster protein, cavortin.

FIG. 4 shows UV absorbance of cavortin purified by high speed centrifugation of cell-free haemolymph and resuspension of the resultant pellet in buffer. The concentration of cavortin in the plasma was estimated by extrapolating from the above concentration to the original volume of plasma. By reference to the values obtained from a standard concentration of bovine serum albumin, the concentration of cavortin in oyster haemolymph was estimated to be 1.17 milligrams per ml.

FIG. 5 is a graph showing the iron content (in nanomoles) of a solution of iron, estimated as described in materials and methods. The trendline equation relates absorbance at 562 nm wavelength to the iron level present in a series of dilutions of ferrous ammonium sulphate. The content of iron bound to cavortin was estimated by reference to a similar plot from each experiment.

### DESCRIPTION OF THE INVENTION

As broadly outlined above, in one aspect the present invention provides a novel protein.

The protein of the invention has an apparent molecular weight of 31 kDa, calculated by polyacrylamide gel electrophoresis (SDS-PAGE).

The protein includes an amino acid sequence which includes one or more of the following:

| | | |
|---|---|---|
| (a) | TAXNEANVNIYLHLSDDEDSNYENS | (SEQ ID NO:1) |
| | (N-terminus) | |
| (b) | EPNAFMPGNLHHRV | (SEQ ID NO:2) |
| (c) | EHGXDTIGEL | (SEQ ID NO:3) |

or an active fragment thereof.

In sequence (a) “X” represents either an “S” or “R” residue reflecting variance in the sequence in the proteins obtained containing same. In sequence (c) “X” represents an inferred “C” residue from microsequencing.

One specific protein of the invention was initially identified as an extract from the Pacific oyster C. gigas. It is therefore obtainable by extraction directly from C. gigas, particularly the haemolymph.

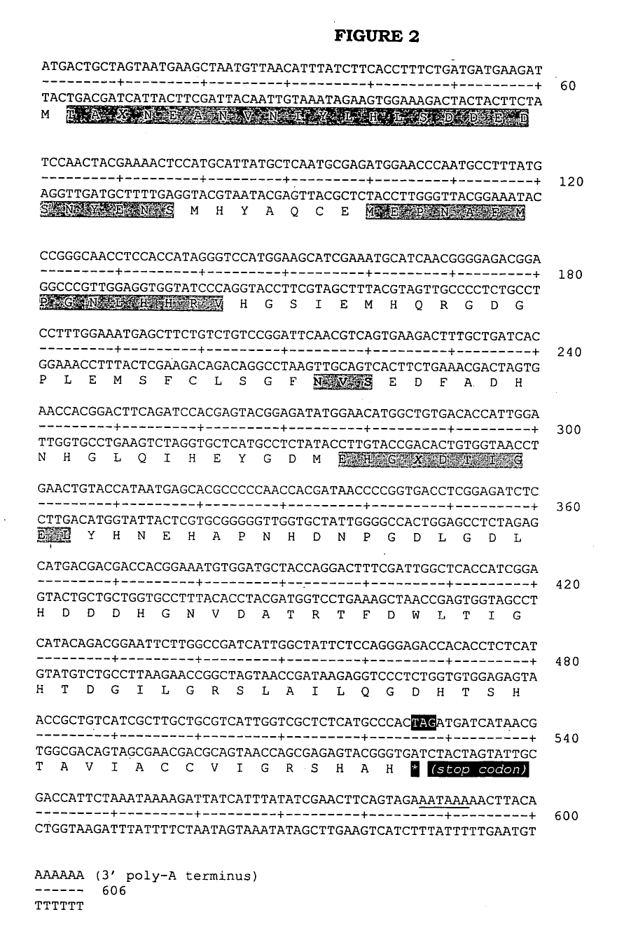

This protein includes the amino acid sequence of SEQ ID NO:4 and/or as shown in FIG. 2 and SEQ ID NO:7.

The molecular mass due to amino acids, inferred from sequencing cDNA derived from mRNA of this protein is 19357 Da. There is one potential glyscosylation site apparent in this sequence, the sequence NVS in blocked print in FIG. 2. This means that a polymer of carbohydrate residues (glycosyl units) is likely attached to the asparagine (N) in the native molecule. This additional mass imparts additional molecular weight to the molecule ˜2 kDa and accounts for some of the variation between predicted and actual molecular weight.

The protein of the invention (also referred to herein as cavortin) can include its entire native amino acid sequence or can include only parts of that sequence where such parts constitute fragments which remain biologically active (active fragments). Such activity will normally be as a metal ion binding agent, especially a divalent cation binding agent, but is not restricted to this activity.

The invention also includes within its scope functionally equivalent variants of the protein of SEQ ID NO:4 and SEQ ID NO:7. This may include a protein or fragment thereof which is obtainable from a shellfish other than Crassostrea gigas and which is a functionally equivalent variant of a protein or fragment of SEQ ID NO:4 and SEQ ID NO:7.

The phrase “functionally equivalent variants” recognises that it is possible to vary the amino acid of a protein while retaining substantially equivalent functionality. For example, a protein can be considered a functional equivalent of another protein for a specific function if the equivalent peptide is immunologically cross-reactive with and has at least substantially the same function as the original protein. The biological activity (e.g. metal ion binding capability) of a protein analog is at least about 25% of a protein of the invention, preferably at least about 50%, preferably at least about 75%, and more preferably at least about 95%.

The functionally equivalent protein need not be the same size as the original. The equivalent can be, for example, a fragment of the protein, a fusion of the protein with another protein or carrier, or a fusion of a fragment with additional amino acids. Active fragments may be obtained by deletion of one or more amino acid residues of the full-length protein. It is also possible to substitute amino acids in a sequence with equivalent amino acids using conventional techniques. Groups of amino acids normally held to be equivalent are:

(a) Ala, Ser, Thr, Pro, Gly;

(b) Asn, Asp, Glu, Gln;

(c) His, Arg, Lys;

(d) Met, Leu, Ile, Val; and

(e) Phe, Tyr, Trp.

Polypeptide sequences may be aligned, and percentage of identical amino acids in a specified region may be determined against another sequence, using computer algorithms that are publicly available. The similarity of polypeptide sequences may be examined using the BLASTP algorithm. BLASTP software is available on the NCBI anonymous FTP server (ftp://ncbi.nlm.nih.gov) under /blast/executables/. The use of the BLAST family of algorithms, including BLASTP, is described at NCBI's website at URL http://www.ncbi.nlm.nih.gov/BLAST/newblast.html and in the publication of Altschul, Stephen F., et al. (1997), “Gapped BLAST and PSI-BLAST: a new generation of protein database search programs”, Nucleic Acids Res. 25:3389-34023. In terms of homology, the polypeptides preferably have at least about 70% homology, more preferably at least about 80% homology, more preferably at least about 90% homology, and even more preferably at least about 95% homology with the protein of SEQ ID NO:4.

Polypeptides of the invention also include homologous polypeptides having an amino acid sequence with at least 55% identity to cavortin (SEQ ID NO:4), preferably at least about 60% identity, preferably at least about 70% identity, more preferably at least about 80% identity, more preferably at least about 90% identity, as well as those polypeptides having an amino acid sequence at least about 95% identical to the protein of SEQ ID NO:4.

A protein of the invention together with its active fragments and other variants may be generated by recombinant or synthetic means (i.e. single or fusion polypeptides). Synthetic polypeptides having fewer than about 100 amino acids, and generally fewer than about 50 amino acids, may be generated by techniques well known to those of ordinary skill in the art. For example, such peptides may be synthesised using any of the commercially available solid-phase techniques such as the Merryfield solid phase synthesis method, where amino acids are sequentially added to a growing amino acid chain (see Merryfield, J. Am. Chem. Soc 85: 2146-2149 (1963)). Equipment for automative synthesis of peptides is commercially available from suppliers such as Perkin Elmer/Applied Biosystems, Inc. and may be operated according to the manufacturers instructions.

A protein of the invention, or a fragment or variant thereof, may also be produced recombinantly by inserting a polynucleotide (usually DNA) sequence that encodes the protein into an expression vector and expressing the protein in an appropriate host. Any of a variety of expression vectors known to those of ordinary skill in the art may be employed. Expression may be achieved in any appropriate host cell that has been transformed or transfected with an expression vector containing a DNA molecule which encodes the recombinant protein. Suitable host cells includes prokaryotes, yeasts and higher eukaryotic cells. Preferably, the host cells employed are E. coli, yeasts or a mammalian cell line such as COS or CHO, or an insect cell line, such as SF9, using a baculovirus expression vector. The DNA sequence expressed in this matter may encode the naturally occurring protein, fragments of the naturally occurring protein or variants thereof.

DNA sequences encoding the protein or fragments may be obtained by screening an appropriate C. gigas cDNA or genomic DNA library for DNA sequences that hybridise to degenerate oligonucleotides derived from partial amino acid sequences of the protein. Suitable degenerate oligonucleotides may be designed and synthesised by standard techniques and the screen may be performed as described, for example, in Maniatis et al. Molecular Cloning—A Laboratory Manual, Cold Spring Harbour Laboratories, Cold Spring Harbour, N.Y. (1989). The polymerase chain reaction (PCR) may be employed to isolate a nucleic acid probe from genomic DNA, a cDNA or genomic DNA library. The library screen may then be performed using the isolated probe.

Variants of the protein may be prepared using standard mutagenesis techniques such as oligonucleotide-directed site specific mutagenesis.

A specific polynucleotide of the invention includes the following nucleotide sequence:

| | |
|---|---|
| ATGACTGCTAGTAATGAAGCTAATGTTAACATTTATCTTCACCTTTCTGATGATGAAGAT | (SEQ ID NO:6) |
| TCCAACTACGAAAACTCCATGCATTATGCTCAATGCGAGATGGAACCCAATGCCTTTATG | |
| CCGGGCAACCTCCACCATAGGGTCCATGGAAGCATCGAAATGCATCAACGGGGAGACGGA | |
| CCTTTGGAAATGAGCTTCTGTCTGTCCGGATTCAACGTCAGTGAAGACTTTGCTGATCAC | |
| AACCACGGACTTCAGATCCACGAGTACGGAGATATGGAACATGGGTGTGACACCATTGGA | |
| GAACTGTACCATAATGAGCACGCCCCCAACCACGATAACCCCGGTGACCTCGGAGATCTC | |
| CATGACGACGACCACGGAAATGTGGATGCTACCAGGACTTTCGATTGGCTCACCATCGGA | |
| CATACAGACGGAATTCTTGGCCGATCATTGGCTATTCTCCAGGGAGACCACACCTCTCAT | |
| ACCGCTGTCATCGCTTGCTGCGTCATTGGTCGCTCTCATGCCCACTAG | |

A further polynucleotide includes the sequence as follows:

| | |
|---|---|
| ATGACTGCTAGTAATGAAGCTAATGTTAACATTTATCTTCACCTTTCTGATGATGAAGAT | (SEQ ID NO:5) |
| TCCAACTACGAAAACTCCATGCATTATGCTCAATGCGAGATGGAACCCAATGCCTTTATG | |
| CCGGGCAACCTCCACCATAGGGTCCATGGAAGCATCGAAATGCATCAACGGGGAGAGGGA | |
| CCTTTGGAAATGAGCTTCTGTGTGTCCGGATTCAACGTCAGTGAAGACTTTGCTGATCAC | |
| AACCACGGACTTCAGATCCACGAGTACGGAGATATGGAACATGGCTGTGACAGCATTGGA | |
| GAACTGTACCATAATGAGCACGCCCCCAACCACGATAACCCCGGTGACCTCGGAGATCTC | |
| CATGACGACGACCACGGAAATGTGGATGCTACCAGGACTTTCGATTGGCTCACCATCGGA | |
| CATACAGACGGAATTCTTGGCCGATCATTGGCTATTCTCCAGGGAGACCACAGCTGTCAT | |
| ACCGCTGTCATGGCTTGCTGCGTCATTGGTCGCTCTCATGCCCACTAGATGATCATAACG | |
| GACCATTCTAAATAAAAGATTATCATTTATATCGAACTTCAGTAGAAATAAAAACTTACA | |
| AAAAAA... (3′ poly-A terminus) | |

with TAG being the stop codon, and AATAAA is the polyadenylation signal. AAAAAA is the beginning of the 3′ poly-A tail.

Variants or homologues of the above polynucleotide sequences also form part of the present invention. Polynucleotide sequences may be aligned, and percentage of identical nucleotides in a specified region may be determined against another sequence, using computer algorithms that are publicly available. Two exemplary algorithms for aligning and identifying the similarity of polynucleotide sequences are the BLASTN and FASTA algorithms. The BLASTN software is available on the NCBI anonymous FTP server (ftp://ncbi.nlm.nih.gov) under /blast/executables/. The BLASTN algorithm version 2.0.4 [Feb-24-1998], set to the default parameters described in the documentation and distributed with the algorithm, is preferred for use in the determination of variants according to the present invention. The use of the BLAST family of algorithms, including BLASTN, is described at NCBI's website at URL http://www.ncbi.nlm.nih.gov/BLAST/newblast.html and in the publication of Altschul, Stephen F, et al (1997). “Gapped BLAST and PSI-BLAST: a new generation of protein database search programs”, Nucleic Acids Res. 25:3389-3402. The computer algorithm FASTA is available on the Internet at the ftp site ftp://ftp.virginia.edu.pub/fasta/. Version 2.0u4, February 1996, set to the default parameters described in the documentation and distributed with the algorithm, is preferred for use in the determination of variants according to the present invention. The use of the FASTA algorithm is described in the W R Pearson and D. J. Lipman, “Improved Tools for Biological Sequence Analysis,” Proc. Natl. Acad. Sci. USA 85:2444-2448 (1988) and W. R. Pearson, “Rapid and Sensitive Sequence Comparison with FASTP and FASTA,” Methods in Enzymology 183:63-98 (1990).

The invention also includes isolated nucleic acid molecules or polynucleotides that comprise a polynucleotide sequence having at least about 55% identity, preferably at least about 60% identity, preferably at least about 70% identity, more preferably at least about 80% identity, more preferably at least about 90% identity, as well as those polynucleotides having a nucleic acid sequence at least about 95% identical to either of the polynucleotide sequences of the invention above (SEQ ID NO:6 and SEQ ID NO:7).

All sequences identified as above qualify as “variants” as that term is used herein.

Variant polynucleotide sequences will generally hybridize to the recited polynucleotide sequence under stringent conditions. As used herein, “stringent conditions” refers to prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C. Such hybridizable sequences include those which code for the equivalent protein from sources (such as shellfish) other than C. gigas.

While the above synthetic or recombinant approaches can be taken to produce the protein of the invention, it is however practicable (and indeed presently preferred) to obtain the protein by isolation from C. gigas. This reflects the applicants' finding that the protein is the dominant protein of the haemolymph of C. gigas and also that the protein is self-aggregating. It can therefore be isolated in commercially significant quantities direct from the oyster itself. For example, approximately 1 mg of the protein can be obtained per millilitre of haemolymph. The haemolymph can be obtained simply by opening the shells of the oyster, draining the initial fluid, and collecting the subsequent fluid by allowing the oysters to drain into a suitable container. In this manner up to 6 ml of haemolymph can be collected from each oyster.

Once obtained, the protein of the invention is readily purified if desired. This will generally involve centrifugation in which the self-aggregating nature of the protein is important. Other approaches to purification (eg. chromatography) can however also be followed.

Furthermore, if viewed as desirable, additional purification steps can be employed using approaches which are standard in this art. These approaches are fully able to deliver a highly pure preparation of the protein. Preferably, the protein preparation comprises at least about 50% by weight of the protein, preferably at least about 80%, preferably at least about 90%, and more preferably at least about 95% by weight of the protein.

In another embodiment, the protein employed in the invention is provided in the form of a shellfish extract. Extracts may be produced simply by liquifying whole shellfish, with or without shells, in an aqueous medium followed by the optional steps of drying and powdering. A process useful for the preparation of such extracts is disclosed in Jones et al. 1996.

The applicants have also unexpectedly found that cavortin and equivalent or related self-aggregating shellfish proteins and extracts can be “loaded” with additional metal molecules in excess of one molecule of metal per molecule of protein. More particularly, the applicants have determined that cavortin can bind up to about 12 molecules of metal per molecule of protein. Of the metal bound, it has been found that for cavortin about four to six molecules of metal are tightly bound.

In contrast, cavortin in its natural state is associated with only low levels of iron—approximately one molecule of iron to every four to six molecules of proteins.

The term “metal enriched” as used herein therefore refers to a protein or extract of the invention or related protein loaded with one or more molecules of metal per molecule of protein.

Preferred self-aggregating shellfish proteins are histidine-rich.

It should be noted that metal enrichment does not require purified protein.

Metal enrichment of a protein or extract herein can be achieved simply by adding a metal of interest to a solution containing the protein, or to the extract. The metal may conveniently be added in the form of a salt or other suitable forms known to those skilled in the art. The added metal ions are bound to the protein molecules, increasing the metal content of the protein beyond the natural level found in the shellfish.

Metals suitable for addition to the proteins and extracts include lead, arsenic, mercury, magnesium, cadmium, zinc, calcium, selenium and iron. Generally, the metals are added in the form of metal ions including divalent cations. Where a metal other than iron is to be added, the proteins may be able to be stripped or partially stripped of existing metals (for example, by pH variation) before adding the metal or metals of interest.

Once obtained, the protein and/or its active fragments or combinations thereof can be formulated into a composition. The composition can be, for example, an agricultural composition, a therapeutic composition for application as a pharmaceutical, or neutraceutical, or can be a health or dietary supplement. For these purposes it is generally preferred that the protein be present in a pure or substantially pure form. Again, standard approaches can be taken in formulating such compositions (see for example, Remington's Pharmaceutical Sciences, 18^{th} Edition, Mack Publishing (1990)).

In one embodiment, the composition is a neutraceutical comprising a protein or extract of the invention and a carrier, diluent or excipient. Suitable carriers, diluents and excipients are known in the art and include water, saline, sugar solutions, oils and the like. Also included may be preservatives, buffers, stabilisers and the like, all of which are also well known in the art.

In a preferred embodiment, the proteins and compositions can be used to bind metal ions facilitating cation recovery and/or bioremediation, for example of soils and solutions. Similarly, proteins and compositions can be formulated with pre-selected metal ions for use in the food and nutraceutical industries.

Still further, the composition can be a food in which the protein and/or its active fragments are included. This can occur by adding the protein to a pre-prepared foodstuff, or incorporating the protein into a step of the manufacturing process for the food.

The invention will now be described more fully in the following experimental section which is provided for illustrative purposes only.

### EXAMPLES

Section 1

A. Materials and Methods

A.1 Shellfish: Crassostrea gigas (the Pacific Oyster were obtained from retail outlets or commercial oyster farms.

A.2 Extracts: The plasma protein from the Pacific oyster (Crassostrea gigas) was obtained by opening the oysters, discarding the initial fluid present, and then collecting the subsequent fluid that appeared by placing the oysters in a funnel and draining the liquid haemolymph into a beaker. Haemocytes were removed by low speed centrifugation and the supernatant (plasma; cell-free haemolymph) was centrifuged at high-speed (eg. 50,000 rpm in a Beckman 60Ti rotor for 60-80 minutes) to produce a pellet consisting solely or predominantly of cavortin. Resuspension in a buffer such as 100 mM sodium phosphate at pH 7.2 or Tris-Cl or any other suitable buffer produced a high concentration of cavortin. Further purification steps could include CsCl isopycnic equilibirum centrifugation, controlled-pore glass chromotography, or using an HPLC system.

A.3 Polyacrylamide gel electrophoresis: 10% polyacrylamide gels (8×10 cm; 1 mm thick) were cast using a prepared stock solution according to the manufacturer's instructions (40% acrylamide/bis solution 37.5:1, Bio-Rad, USA); commercially available 12% gels (Bio-Rad, USA) were also used. Samples (10 μl) were applied to lanes and the gels run at 160 V using a standard Tris/Glycine/SDS buffer (Bio-Rad, catalogue 161-0732) until the bromphenol blue marker reached the bottom of the gel. Gels were stained with BM Fast Stain Coomassie® (Boehringer Mannheim, Germany) and destained as per the manufacturer's instructions.

A.4 Isopycnic gradients: CsCl (Boehringer Mannhein, Germany) solutions were prepared in 0.1 M sodium phosphate buffer, pH 7.2 and filtered through a 0.22 μm membrane (Acrodisc, Gelman Sciences, USA) to clarify. Two step gradients (1.25 g/cc top layer containing the sample and 1.45 g/cc bottom layer) were prepared as described by Scotti (1985) and centrifuged for approximately 17 hours at 20° C. in a Beckman 70Ti rotor at 30,000 rpm. The resultant gradient was fractionated by inserting a small diameter tube into the gradient and slowly pumping out the contents. UV absorbance was monitored by passing through a Uvicord spectrophotometer (LKB Produkter, Sweden) or monitoring collected fraction by UV spectrophotometry. Fractions were collected and the refractive indices measured using an Abbé refractometer (Bellingham and Stanley, UK) and the density estimated using regression equations according to the method of Scotti (1985).

A.5 Porous glass chromatography: Controlled pore glass (CPG 240-80, Sigma Chemical Co., USA) was treated according to the suppliers directions. A 1 cm×100 cm column (Bio-Rad, USA) was prepared. Samples (1-2 ml) were loaded onto the column and eluted with 0.1 M sodium phosphate buffer, pH 7.2, through a Uvicord spectrophotometer, fractions being collected at regular intervals.

A.6 Estimation of protein concentration: Concentrations were estimated using by UV absorption according to the method of Layne (1957) using the basic equation: mg/ml protein=1.55*A280−0.76*A260. Highly-purified, freeze dried cavortin was weighed and redissolved in a known volume of water or buffer. The relationship between the Layne equation and the actual concentration was corrected by including an appropriate factor. The concentration of cavortin could also be estimated using the extinction coefficients estimated from the inferred amino acid sequence according to a program (available through www.up.univ-mrs.fr/cgi-wabim): the 0.1% (mg/ml) absorption, taking into account cysteines, was 0.640. This value approximated the value obtained by direct weighing and estimating using the Layne equation. Alternatively, concentration was estimated spectrophotometrically at a wavelength of 595 nm using the Protein Assay Reagent supplied by Bio-Rad (USA) according to the supplier's directions, standardising the absorbance values to known concentrations of cavortin as described above.

A.7 High performance liquid chromatography: Reversed-phase HPLC was performed on an HP 1050 Ti-series HPLC (Hewlett Packard, USA) fitted with an analytical 300 Å Vydac C-18 column, 25 cm×4.6 mm i.d. The 10 μl sample in water containing 0.1% trifuoroacetic acid (TFA) was eluted with a 0-100% acetonitrile in water (v/v) gradient containing 0.1% TFA over 60 min and the absorption at 218 nm was recorded.

A.8 Gene Sequencing Method

A suite of non-specific primers called OZ2 was synthesised by Gibco-BRL for the initial sequencing based on an internal sequence (MEPNAFMPGNL) of cavortin obtained by microsequencing fragments following CNBr cleavage of mature cavortin. The general formula was:

ATGCCNAAYGCNTTYATGCCNGGNAA.

Where Y represents a pyrimidine base, and N represents any one of the four nucleotide bases. Sequencing was done, initially using OZ2 and an oligo-dT based “bottom strand” (=“reverse strand”) primer to produce a PCR product from the cDNA. This was electrophoresed on a 1% agarose gel under standard conditions in a tris-based buffer. The approximately 550 base pair band observed after ethidium bromide stain was excised and cloned into an E. coli vector and amplified by culture in broth medium. Sequencing of this cavortin-based insert was by dye-termination cycle sequencing using “BigDye” prism technology (Applied Biosystems Incorporated, USA) according to their instructions. Products were resolved on an ABI 377 automated sequencer.

This provided the following:

| | |
|---|---|
| ATGACTGCTAGTAATGAAGCTAATGTTAACATTTATCTTCAGCTTTCTGATGATGAAGAT | (SEQ ID NO:5) |
| TCCAACTACGAAAACTCCATGCATTATGCTCAATGCGAGATGGAACCCAATGCCTTTATG | |
| CCGGGCAACCTCCACCATAGGGTCCATGGAAGCATCGAAATGCATCAACGGGGAGACGGA | |
| CCTTTGGAAATGAGCTTCTGTCTGTCCGGATTCAAGGTCAGTGAAGACTTTGCTGATCAC | |
| AACCACGGACTTCAGATCCACGAGTACGGAGATATGGAACATGGCTGTGACACCATTGGA | |
| GAACTGTACCATAATGAGCACGCCCCCAACCACGATAACCCCGGTGACCTCGGAGATCTC | |
| CATGACGACGACCACGGAAATGTGGATGCTACCAGGACTTTCGATTGGCTCACCATCGGA | |
| CATACAGACGGAATTCTTGGCCGATCATTGGCTATTCTCCAGGGAGACCACACCTCTCAT | |
| ACCGCTGTCATCGCTTGCTGCGTCATTGGTCGCTCTGATGCCCACTAGATGATCATAACG | |
| GACCATTCTAAATAAAAGATTATCATTTATATCGAACTTCAGTAGAAATAAAAACTTACA | |
| AAAAAA | |

A9: Metal Binding

Materials and Methods

One ml capacity Hi Trap® chelating affinity columns (Amersham Pharmacia Biotech, UK) were prepared according to the manufacturer's instructions. The columns were charged with 500 ml of 100 mM solutions of several metal salts (anhydrous cupric chloride; cobaltous chloride hexahydrate; nickel chloride hexahydrate; zinc chloride) in water and subsequently washing and equilibrating the column in buffer (0.05 M sodium phosphate, pH 7.2 containing 500 mM sodium chloride). Cavortin, purified by several cycles of ultracentrifugation, was suspended in this buffer and approximately 1 mg was applied slowly to the column using a syringe.

To determine the binding of cavortin to copper the column was first washed with 5 ml of buffer and then 5 ml of buffer containing 200 mM imidazole. With the nickel and zinc-charged columns 50 mM disodium EDTA replaced imidazole in the elution buffer. The absorbance of fractions was monitored at 280 nm using a Pye Unicam SP1800 spectrophotometer. For the cobalt-charged column, elution was performed using buffers containing 200 mM imidazole, 500 mM imidazole and 50 mM disodium EDTA. All elution buffers were adjusted to pH 7.2.

A.10 Assay for protein bound iron: A sensitive assay for the determination of the iron content of cavortin was used (Davis, M D, Kaufman, S and Milstien, S. (1986) A modified ferrozine method for the measurement of enzyme-bound iron. J. Biochem Biophys Methods 13, 39-45). Ferrous ammonium sulphate dissolved in ultra-pure water was used as the standard. A standard linear plot of nmoles of iron vs absorbance at 562 nm, ranging from 99 nmoles to 2 nmoles of iron as well as a blank, were determined for each experiment. Concentrated methanesulfonic acid (15.4 M) was used to allow for greater amounts of sample protein material to be assayed.

A.11 Measurement of the binding capacity of the shellfish protein for iron: Ferrous ammonium sulphate hexahydrate was dissolved in ultra pure water at a known concentration. Aliquots were added to a solution of purified cavortin at various ratios (“iron-loading”). Excess (unbound) iron was removed by centrifugation through a gel filtration column (BioRad Micro Bio-Spin P-6 column cat #732-6222). Columns were loaded with up to 70 μl of sample and treated according to the manufacturer's directions and the filtrate analysed for iron content as described above. The molar ratio of iron (“bound iron”) to protein was calculated by estimating the moles of protein present based upon the methods described above. To examine the strength of the binding of iron to protein, iron-loaded cavortin were treated with disodium EDTA at an excess of EDTA to iron. EDTA and unbound iron were subsequently removed by using a BioSpin P-6 column or alternatively by exhaustive dialysis against water or a suitable buffer.

B. Results

A light-scattering band was observed after centrifugation of oyster plasma in CsCl isopycnic gradients. The density of this band was estimated as 1.37-1.38 g/cc.

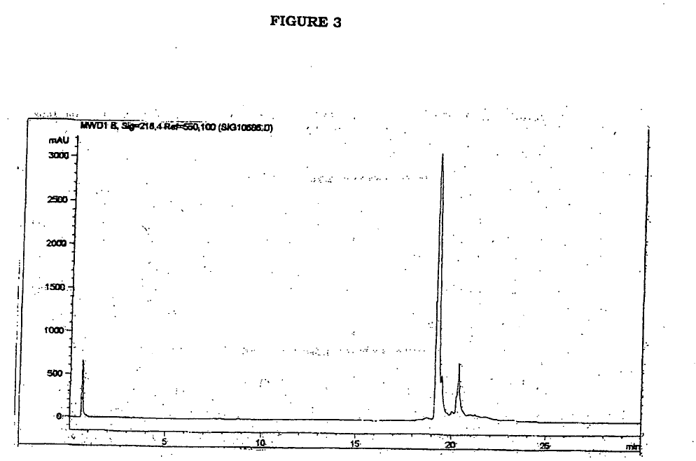

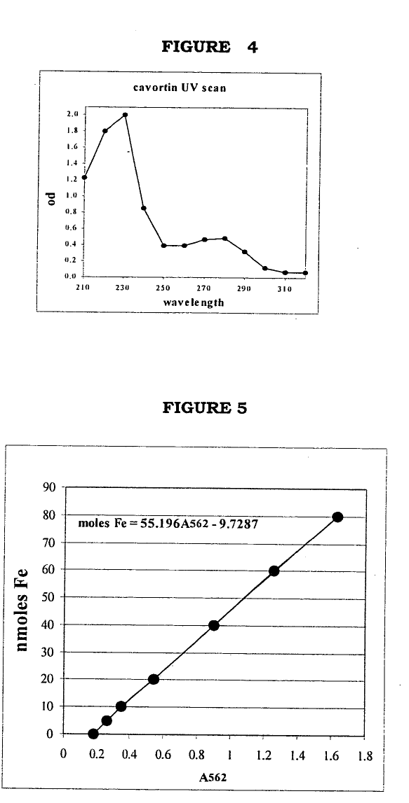

Chromatography, on a CPG 240-80 column, of semi-purified extracts, or of material banded in CsCl, showed that the majority of cavortin eluted in the exclusion volume using low molarity phosphate or Tris buffer (usually 100 mM buffers) as the eluents. In contrast, a protein of greater molecular mass, bovine serum albumin (68 kDa), was included in the column matrix. It appears, therefore, that cavortin aggregates into large, particle-like structures. HPLC confirmed that the cavortin from C. gigas obtained by CPG chromatography was highly purified (see FIG. 3).

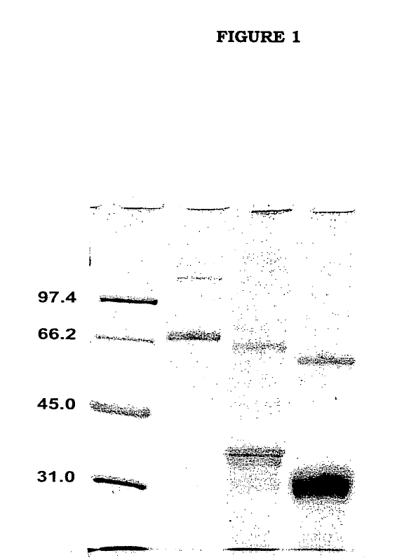

The yield of cavortin averaged about 1 mg/ml of haemolymph which was obtained directly from oysters by opening the shell, discarding the initial fluid, and collecting the subsequent fluid. The haemolymph was spun at low speed (≈1000 g) to remove haemocytes and the resulting supernatant processed by ultracentrifugation, for example at 250,000 g for 40 minutes, followed by either CPG chromatography eluting with 0.1 M sodium phosphate buffer, pH 7.2, or isopycnic banding in CsCl in phosphate buffer. Haemolymph contained around 1 mg/ml (average ≈5-6 ml of haemolymph per oyster) of cavortin which is by far the most predominant polypeptide species (see FIG. 1 and FIG. 3).

Microsequencing of the N-terminal region and internal fragments generated by chemical and enzymatic cleavage from purified cavortin was performed and generated the following sequences of cleavage fragments:

(a) TAXNEANVNIYLHLSDDEDSNYENS (N-terminus)

(b) EPNAFMPGNLHHRV

(c) EHGXDTIGEL

These sequences code for amino acids as follows:

| CODE: | |
|---|---|
| A | alanine |
| C | cystine |
| D | aspartic acid |
| E | glutamic acid |
| F | phenylalanine |
| G | glycine |
| H | histidine |
| I | isoleucine |
| K | lysine |
| L | leucine |
| M | methionine |
| N | asparagine |
| P | proline |
| Q | glutamine |
| R | arginine |
| S | serine |
| T | threonine |
| V | valine |
| W | tryptophan |
| Y | tyrosine |

Each of fragments (a) to (c) above are part of the larger cavortin amino acid sequence:

| | |
|---|---|
| TAXNEANVNIYLHLSDDEDSNYENSMHYAQCEMEPNAFMPGNLHHRVHGSIEMH | (SEQ ID NO:4) |
| QRGDGPLEMSFCLSGFNVSEDFADHNHGLQIHEYGDMEHGCDTIGELYHNEHAP | |
| NHDNPGDLGDLHDDDHGNVDATRTFDWLTIGHTDGILGRSLAILQGDHTSHTAVI | |
| ACCVIGRSHAH | |

Natural Association of Cavortin and Pernin with Iron

The quantity of iron associated with shellfish proteins was estimated spectrophoto-metrically using the method of Davis et al (1986) incorporated herein by reference. Cavortin was obtained by the extraction process discussed above. The binding ratio for the oyster protein, cavortin, was estimated at 1 atom of iron to 4.5 molecules of cavortin.

TABLE 1

| Sample | nmoles iron/ml | ratio (moles) iron:protein |
|---|---|---|
| Cavortin ex high speed spin | 14.3 | 1:4.5 |

Metal Binding

The results from Hi Trap® chelating affinity columns showed that cavortin bound to copper, zinc, cobalt and nickel. No UV absorbing material (above background) was detected in the eluant following washing of the column with 5 ml of buffer. However, all the UV-absorbing material (protein) eluted in the first two ml of eluant using buffer containing imidazole for copper or EDTA for zinc and nickel (no imidazole buffer was tried for these). With the cobalt-charged column, no cavortin was eluted with 200 mM imidazole buffer, but approximately 40% of the protein eluted with 500 mM imidazole and the remaining protein eluted with EDTA-containing buffer.

Iron-Loading

The iron assay is highly sensitive and can readily detect 1-2 nmoles of iron (FIG. 5). For each experiment, a 100 mM solution of ferrous ammonium sulphate was appropriately diluted and the regression equation (“trendline”) calculated as a reference standard for measurement of the amount of iron bound to pernin or cavortin.

Cavortin in haemolymph is naturally associated with low levels of iron (Scotti et al. 2001). The concentration of cavortin in oyster plasma is approximately 1 mg/ml. These estimates are based upon the population of the proteins observed in haemolymph and indicate that only a small proportion of the cavortin present in shellfish contains bound iron.

TABLE 2
Estimated levels of iron in plasma (cell-free haemolymph)

| Sample | nmoles Fe/ml | nmoles protein/ml | Ratio (Fe/protein) |
|---|---|---|---|
| oyster plasma | 5.7 | 51.4 | 0.1 |

The shellfish protein can be loaded with iron by adding, for example, ferrous ammonium sulphate (a salt) to a solution of cavortin. The results (Table 3) indicate that each molecule of cavortin can bind up to 10 or 11 molecules of iron with perhaps 4 to 6 being tightly bound.

TABLE 3
Iron loading of cavortin

| Sample | nmoles iron bound | ratio (bound iron/protein) |
|---|---|---|
| Cavortin, no iron added | n.d. | n.d. |
| Cavortin + iron | 115.2 | 10.3 |
| Cavortin + iron + EDTA | 53.9 | 4.8 |

The basic method is as described in Materials and Methods using BioSpin P-6. Iron (a solution of ferrous ammonium sulphate hexahydrate) was added to a solution of cavortin (10 nmoles) at a molecular ratio of 20:1 (i.e. a 20-fold excess of iron). For EDTA treatment, iron-loaded cavortin was subsequently treated by adding EDTA in a ratio of 1.9 to 1 (EDTA:iron). Estimate of cavortin concentration by corrected extinction coefficient (ref 02-001 and corrected spreadsheet) new estimate conc=35.45—these were correlated by weighed cavortin experiment data ref 02-001)—previous estimate of conc of cavortin was 32 mg/ml and used “10 nmoles” per tube so new nmoles of cavortin is 11.14780 nmoles per tube; “n.d.” means no detectable iron in sample.

It should be noted that iron loading does not require purified protein. Cavortin can be iron-loaded by adding ferrous ammonium sulphate to a crude aqueous extract of whole mussels provided for example by the method of Jones et al. 1996.

Discussion

The present invention provides a novel protein obtainable from Crassostrea gigas, the Pacific Oyster. The protein appears to be able to self-aggregate into large particles and because of this property it sediments at a relatively high value compared to that normally observed for proteins. The protein was found in extracts of whole oysters and appears to be the predominant protein in haemolymph. The molecular weight of the protein was estimated to be 31 kDa by SDS-PAGE although the weight inferred from the cDNA sequence is only approximately 20 kDa. Because of its ability to aggregate, the protein can be sedimented by ultracentrifugation in a short time (e.g. 40 minutes at 250,000 g) whereas the monomeric protein would not. Each millilitre of haemolymph yields, on the average, about 1 mg of cavortin. Haemolymph is easily obtained by draining the haemolymph from the opened oyster which can yield up to 6 ml. The haemolymph obtained not only contains high levels of cavortin but is quite free of contaminating materials, so purification of cavortin is simple. For highly pure preparations of cavortin, ultracentrifugation can be followed by isopycnic banding in a suitable density gradient medium such as CsCl.

The copper-zinc superoxide dismutase evolutionarily-related sequence cavortin, aggregates into multimeric units forming stable entities at physiological pH and osmotic strength. Cavortin once possessed the ability to bind cations such as Cu++ and Zn++. While some of the critical co-ordinating metal binding ligands (primarily Histidine residues) have been lost at the active site, thereby rendering this protein inactive as a SOD, there is instead a natural level of iron bound to this protein. This level of iron saturation is only some 3% of the total population of cavortin molecules.

Cavortin is naturally associated with iron. However, not all the molecules have iron bound since the ratio of iron to cavortin in the natural is less than 1 (Table 1). Cavortin also has the ability to bind iron as well as other metal ions, e.g copper, zinc, cobalt and nickel.

As demonstrated above cavortin also has the ability to bind significant amounts of metals to produce metal enriched proteins, and extracts.

### INDUSTRIAL APPLICATION

The preferred protein of the invention, cavortin, has a number of utilities.

The cavortin protein from C. gigas as an extract, as a protein per se or in metal enriched form may have value as a pharmaceutical. It may also be useful as a natural therapeutic agent or health supplement particularly where shellfish proteins have value as dietary supplements in their own right. Cavortin is readily obtained as a natural product in high concentrations from oyster haemolymph. To obtain a highly pure preparation it is necessary only to remove haemocytes by centrifugation (or any other suitable method) followed by either ultracentrifugation (since cavortin forms aggregates which readily sediment) and resuspension, isopycnic banding in a suitable medium such as CsCl, exclusion filtration through a suitable membrane which retains cavortin, or chromatography through a medium such as controlled pore glass of suitable porosity. The result is a highly pure preparation of cavortin.

Similarly, obtaining the metal enriched protein is achieved by simply adding a metal of interest, preferably in salt form to a solution containing the protein of extract.

The Pacific oyster C. gigas produces large amounts of the protein cavortin naturally, with little cost or effort involved in production, processing or purification.

Because cavortin molecules can accept metal ions other than iron, e.g. copper, zinc, cobalt and nickel as demonstrated by affinity column chromatography, the protein has potential application as a bioremediator of selected metal ions.

As will be appreciated, for food applications the bound ions are most usually intended to be non-toxic cations such as calcium, magnesium, selenium or zinc, for example.

The ability to bind metal ions, particularly divalent metal cations also gives rise to applications of the protein in bioremediation and/or cation recovery processes. The metal ions or divalent cations such as Pb⁺⁺, As⁺⁺, Hg⁺⁺, Cd⁺⁺ can be present as contaminants or pollutants in media, such as a liquid, solution or solid media. For example, water or soil samples. The solution or sample may be passed by a substrate to which the protein is bound so that the cations are extracted.

Those persons skilled in the art will understand that the above description is provided by way of illustration only and that the invention is not limited thereto.

### REFERENCES

Altschul, Stephen F, et al (1997). “Gapped BLAST and PSI-BLAST: a new generation of protein database search programs”, Nucleic Acids Res. 25:3389-3402

Jones, J. B, Scotti, P. D, Dearing, S. C and Wesney, B. 1996. Virus-like particles associated with marine mussel mortalities in New Zealand. Diseases of Aquatic Organisms 25, 143-149.

Layne, E., 1957. Spectrophotometric and turbidometric methods for measuring proteins. Methods Enzymol. III, 447-455.

Maniatis et al. Molecular Cloning—A Laboratory Manual, Cold Spring Harbour Laboratories, Cold Spring Harbour, N.Y. (1989)

Merryfield, J. Am. Chem. Soc 85: 2146-2149 (1963]

Nair, P. S. and Robinson, W. E. 2001. Histidine-rich glycoprotein in the blood of the bivalve Mytilus edulis: role in cadmium speciation and cadmium transfer to the kidney. Aquatic Toxicology 52, 133-142.

Pearson W R and Lipman, D J “Improved Tools for Biological Sequence Analysis,” Proc. Natl. Acad. Sci. USA 85:2444-2448 (1988)

Pearson, W R “Rapid and Sensitive Sequence Comparison with FASTP and FASTA,” Methods in Enzymology 183:63-98 (1990)

Robinson, W. E., Ryan, D. K., Sullivan, P. A. and Boggs, C. C. 1997. Cadmium binding in the blood plasma of two marine bivalves. Environmental Toxicology and Chemistry 16, 1195-1202.

Scotti, P. D., 1985. The estimation of virus density in isopycnic cesium chloride gradients. J. Virol. Methods 12, 149-160

1. An isolated protein which has a molecular weight of about 31 kDa as determined by SDS-PAGE and an amino acid sequence which includes one or more of the following:

| | | |
|---|---|---|
| (a) | TAXNEANVNIYLHLSDDEDSNYENS | (SEQ ID NO:1) |
| | (N-terminus) | |
| (b) | EPNAFMPGNLHHRV | (SEQ ID NO:2) |
| (c) | EHGXDTIGEL | (SEQ ID NO:3) |

or an active fragment thereof.

2. An isolated protein which comprises the amino acid sequence of SEQ ID NO:4, or an active fragment thereof.

3. An isolated protein as claimed in claim 1 or claim 2, which is obtained from the haemolymph of Crassostrea gigas.

4. An isolated protein or fragment as claimed in any one of claims 1 to 3 which has a molecular weight of approximately 20 kDa as predicted from the cDNA sequence encoding it.

5. A protein or fragment as claimed in any one of claims 1 to 4 which has activity as a metal ion binding agent.

6. A protein or fragment as claimed in claim 5 which has activity as a divalent binding agent.

7. A protein which is a functionally equivalent variant of a protein or fragment as claimed in claim 5 or claim 6, and which has at least about 80% identity with the amino acid sequence of SEQ ID NO:4

8. A protein which is a functionally equivalent variant of a protein or fragment as claimed in claim 5 or claim 6, and which has at least about 90% identity with the amino acid sequence of SEQ ID NO:4

9. A protein which is a functionally equivalent variant of a protein or fragment as claimed in claim 5 or claim 6, and which has at least about 95% identity with the amino acid sequence of SEQ ID NO:4

10. A protein which is obtainable from a shellfish other than Crassostrea gigas, which is a functionally equivalent variant of a protein or fragment as claimed in claim 5 or claim 6 and which has at least about 80% identity with the amino acid sequence of SEQ ID NO:4

11. A protein which is obtainable from a shellfish other than Crassostrea gigas, which is a functionally equivalent variant of a protein or fragment as claimed in claim 5 or claim 6 and which has at least about 90% identity with the amino acid sequence of SEQ ID NO:4

12. A protein which is obtainable from a shellfish other than Crassostrea gigas, which is a functionally equivalent variant of a protein or fragment as claimed in claim 5 or claim 6 and which has at least about 95% identity with the amino acid sequence of SEQ ID NO:4

13. A polynucleotide encoding a protein or fragment as claimed in any one of claims 1 to 12.

14. A polynucleotide as claimed in claim 13 which has the nucleotide sequence of SEQ ID NO:5 or a variant thereof.

15. A polynucleotide which comprises the nucleotide sequence of SEQ ID NO:6 or a variant thereof.

16. A polynucleotide sequence which encodes the amino acid sequence of SEQ ID NO:7 or a variant thereof.

17. A vector which includes a polynucleotide as claimed in any one of claims 13 to 16.

18. A host cell which expresses a polynucleotide as claimed in any one of claims 13 to 16.

19. A composition which comprises a protein or fragment as claimed in any one of claims 1 to 12.

20. A composition as claimed in claim 19 which is a medicament.

21. A composition as claimed in claim 19 which is a food.

22. A composition as claimed in claim 19 which is a dietary supplement.

23. A dietary supplement as claimed in claim 22 in which said protein or fragment is associated with or bound to at least one metal ion of dietary significance.

24. A dietary supplement as claimed in claim 23 wherein said metal ion is a divalent metal cation.

25. A dietary supplement as claimed in claim 24 wherein said divalent metal cation is calcium, magnesium, selenium, or zinc.

26. A composition as claimed in claim 19 which is a bioremediation agent.

27. A process for obtaining a protein as claimed in claim 3 which comprises the step of centrifuging material containing Crassostrea gigas haemolymph or an extract thereof and recovering the sedimented protein.

28. A process as claimed in claim 27 wherein said centrifuging step is ultracentrifugation.

29. A process as claimed in claim 28 wherein said ultracentrifugation is performed for about 40 minutes at about 250,000 g.

30. A process as claimed in any one of claims 27 to 29 which includes the preliminary step of extracting haemolymph from Crassostrea gigas.

31. A metal enriched protein which is a copper/zinc superoxide dismutase derived self-aggregating protein obtainable from shellfish, or a functionally equivalent variant or fragment thereof.

32. A metal enriched protein as claimed in claim 31 which is obtained from shellfish.

33. A metal enriched protein as claimed in any one of claims 31 to 32 which is hisitidine rich.

34. A metal enriched protein as claimed in any one of claims 31 to 33 which is a protein according to any one of claims 1 to 12.

35. A metal enriched protein as claimed in any one of claims 31 to 33 which is obtained from Crassostrea gigas.

36. A protein or fragment as claimed in any one of claims 1 to 12 which is a metal enriched protein or fragment.

37. A shellfish extract containing a metal enriched protein or fragment as claimed in any one of claims 31 to 36.
